(19) United States
(12) Patent Application Publication
(10) Pub. No.: US 2012/0271399 A1
(43) Pub. Date: Oct. 25, 2012

(54) High Metal to Vessel Ratio Landing Zone Stent-Graft and Method

(75) Inventors: Keith Perkins, Santa Rosa, CA (US); Samuel Robaina, Santa Rosa, CA (US); Jeffery Argentine, Petaluma, CA (US); Walter Bruszewski, Windsor, CA (US); Andrew Kiehl, Santa Rosa, CA (US)

(73) Assignee: Medtronic Vascular, Inc., Santa Rosa, CA (US)

(21) Appl. No.: 13/089,803

(22) Filed: Apr. 19, 2011

(51) Int. Cl. A61F 2/84; A61F 2/82

(52) U.S. Cl. 623/1.12; 623/1.15

(57) ABSTRACT

A method includes covering an ostium of a branch vessel emanating from a main vessel with a proximal landing zone of a high metal to vessel ratio landing zone stent-graft, wherein a metal to vessel ratio of the proximal landing zone when deployed is sufficiently high to encourage tissue ingrowth around the proximal landing zone yet is sufficiently low to ensure perfusion of the branch vessel through the proximal landing zone. The method further includes covering an aneurysm of the main vessel with an exclusion zone of the high metal to vessel ratio landing zone stent-graft, the exclusion zone being formed of graft material. By forming the exclusion zone of graft material, excellent exclusion of the aneurysm is achieved.

### BACKGROUND

1. Field

The present application relates to an intra-vascular device and method. More particularly, the present application relates to a device for treatment of intra-vascular diseases.

2. Description of the Related Art

A conventional stent-graft typically includes a radially expandable reinforcement structure, formed from a plurality of annular stent rings, and a cylindrically shaped layer of graft material, sometimes called graft cloth, defining a lumen to which the stent rings are coupled. Main stent-grafts are well known for use in tubular shaped human vessels.

To illustrate, endovascular aneurysmal exclusion is a method of using a stent-graft to exclude pressurized fluid flow from the interior of an aneurysm, thereby reducing the risk of rupture of the aneurysm and the associated invasive surgical intervention.

Stent-grafts with custom side openings are sometimes fabricated to accommodate the particular vessel structure of each individual patient. Specifically, as the location of branch vessels emanating from a main vessel, e.g., having the aneurysm, varies from patient to patient, stent-grafts are fabricated with side openings customized to match the position of the branch vessels of the particular patient. However, custom fabrication of stent-grafts is relatively expensive and time consuming.

Further, the stent-grafts must be deployed such that the custom side openings are precisely aligned with the respective locations of the branch vessels. This is a relatively complex procedure thus increasing the risk of the procedure.

### SUMMARY

A method includes covering an ostium of a branch vessel emanating from a main vessel with a proximal landing zone of a high metal to vessel ratio landing zone stent-graft. The metal to vessel ratio of the proximal landing zone is sufficiently high to encourage tissue ingrowth around the proximal landing zone yet is sufficiently low to ensure perfusion of the branch vessel through the proximal landing zone. The ingrowth of tissue provides secure fixation and sealing of the proximal landing zone to the main vessel thus minimizing the risk of endoleaks and migration.

Further, deployment of the high metal to vessel ratio landing zone stent-graft is relatively simple thus minimizing the complexity and thus risk of the procedure. More particularly, as the entire proximal landing zone is permeably, the high metal to vessel ratio landing zone stent-graft is deployed without having to rotationally position the high metal to vessel ratio landing zone stent-graft to be aligned with the branch vessel.

The method further includes covering an aneurysm of the main vessel with an exclusion zone of the high metal to vessel ratio landing zone stent-graft, the exclusion zone being formed of graft material. By forming the exclusion zone of graft material, excellent exclusion of the aneurysm is achieved.

These and other features of embodiments will be more readily apparent from the detailed description set forth below taken in conjunction with the accompanying drawings.

### BRIEF DESCRIPTION OF THE DRAWINGS

FIG. 1 is a perspective view of a high metal to vessel ratio landing zone stent-graft in its final configuration in accordance with one embodiment;

FIG. 2 is a cross-sectional view of the high metal to vessel ratio landing zone stent-graft of FIG. 1;

FIG. 3 is a cross-sectional view of a vessel assembly including a delivery system containing the high metal to vessel ratio landing zone stent-graft of FIGS. 1 and 2 in accordance with one embodiment;

FIG. 4 is a cross-sectional view of the vessel assembly including the delivery system at a later stage of deploying the high metal to vessel ratio landing zone stent-graft of FIGS. 1 and 2 in accordance with one embodiment;

FIG. 5 is a cross-sectional view of the vessel assembly of FIGS. 3-4 after deployment of the high metal to vessel ratio landing zone stent-graft of FIGS. 1 and 2 in accordance with one embodiment;

FIG. 6 is an enlarged cross-sectional view of the region VI of FIG. 5 illustrating tissue ingrowth into a proximal landing zone of the high metal to vessel ratio landing zone stent-graft in accordance with one embodiment; and

FIG. 7 is a cross-sectional view of the high metal to vessel ratio landing zone stent-graft of FIG. 1 in accordance with another embodiment.

In the following description, the same or similar elements are labeled with the same or similar reference numbers.

### DETAILED DESCRIPTION

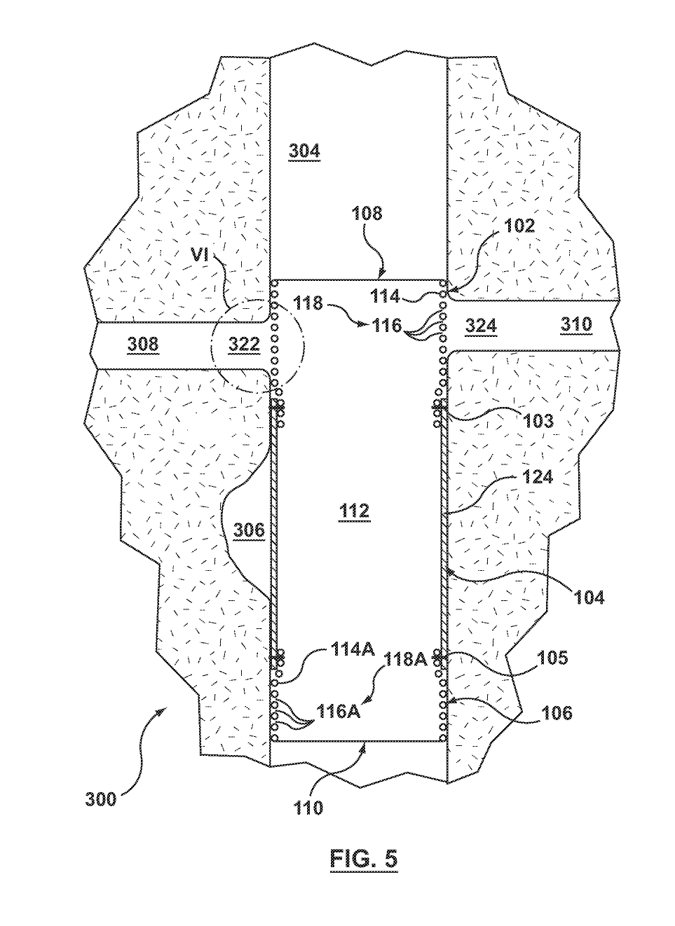

As an overview and in accordance with one embodiment, referring to FIG. 5, a method includes covering ostai 322, 324 of branch vessels 308, 310 emanating from a main vessel 304 with a proximal landing zone 102 of a high metal to vessel ratio landing zone stent-graft 100. A metal to vessel ratio of the proximal landing zone 102 is sufficiently high to encourage tissue ingrowth around proximal landing zone 102 yet is sufficiently low to ensure perfusion of branch vessels 308, 310 through proximal landing zone 102. The ingrowth of tissue provides secure fixation and sealing of proximal landing zone 102 to main vessel 304 thus minimizing the risk of endoleaks and migration.

Further, deployment of high metal to vessel ratio landing zone stent-graft 100 is relatively simple thus minimizing the complexity and thus risk of deploying high metal to vessel ratio landing zone stent-graft 100. More particularly, as the entire proximal landing zone 102 is permeably, high metal to vessel ratio landing zone stent-graft 100 is deployed without having to rotationally position high metal to vessel ratio landing zone stent-graft 100 to be aligned with branch vessels 308, 310.

The method further includes covering an aneurysm 306 of main vessel 304 with an exclusion zone 104 of high metal to vessel ratio landing zone stent-graft 100, exclusion zone 104 being formed of graft material 124. By forming exclusion zone 104 of graft material 124, excellent exclusion of aneurysm 306 is achieved.

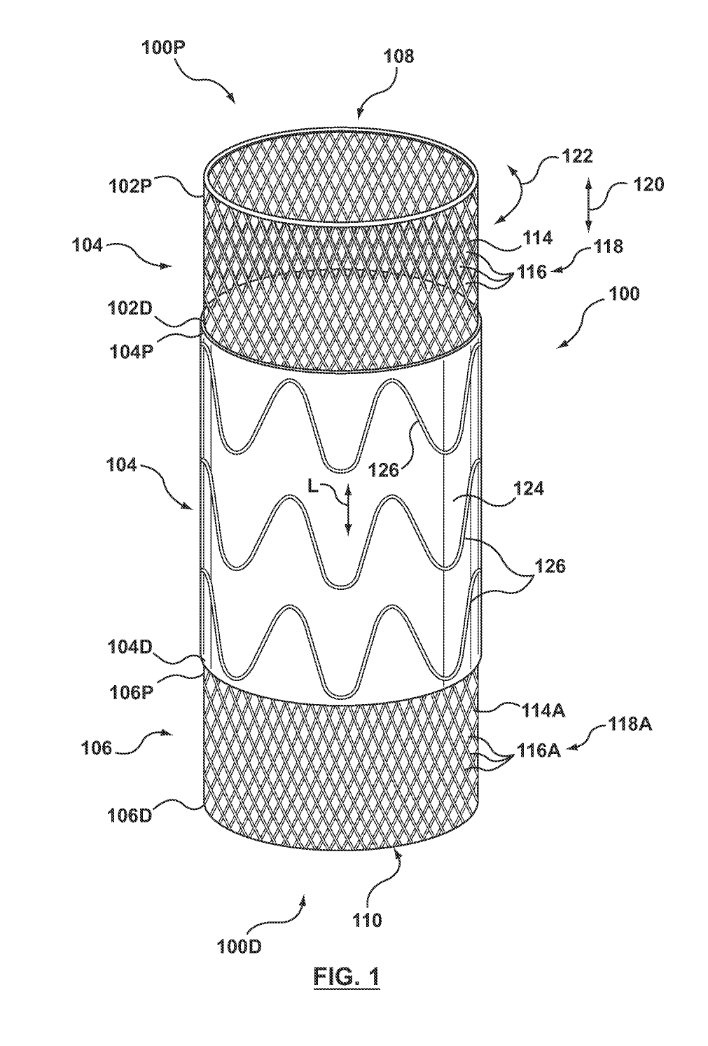

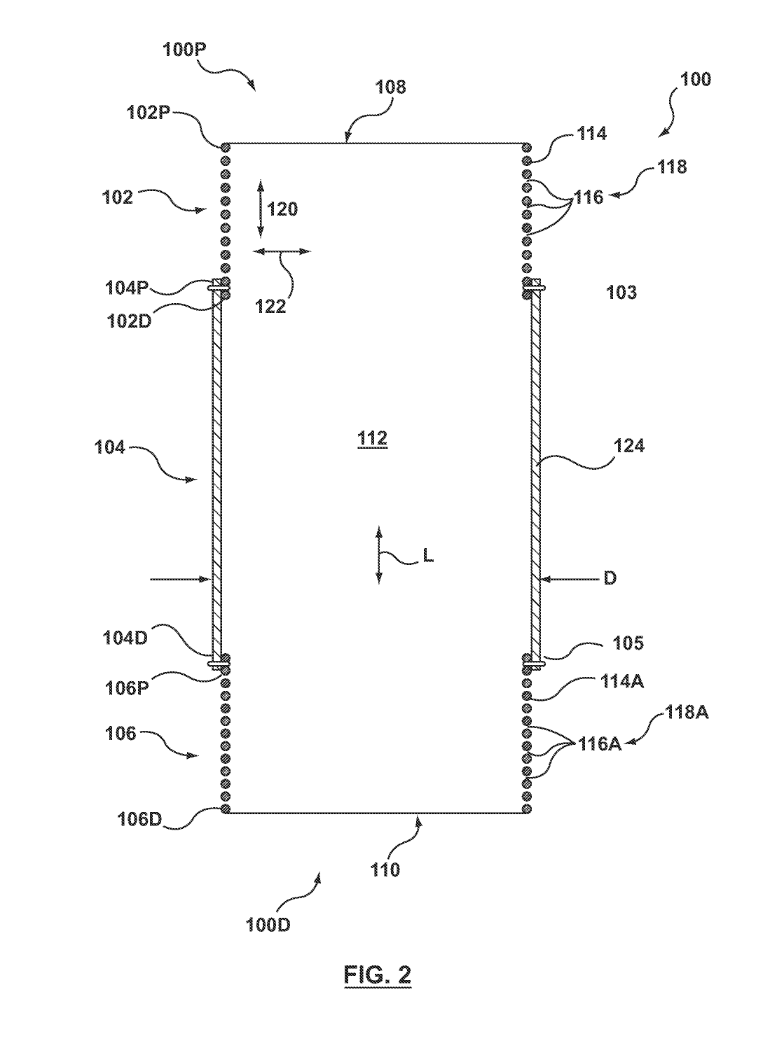

Now in more detail, FIG. 1 is a perspective view of a high metal to vessel ratio landing zone stent-graft 100, e.g., an abdominal aortic stent-graft, in its final configuration in accordance with one embodiment. FIG. 2 is a cross-sectional view of high metal to vessel ratio landing zone stent-graft 100 of FIG. 1. High metal to vessel ratio landing zone stent-graft 100 is sometimes called an endoluminal flow disrupting device.

Referring now to FIGS. 1 and 2 together, high metal to vessel ratio landing zone stent-graft 100 includes a proximal landing zone 102, an exclusion zone 104, and a distal landing zone 106. Exclusion zone 104 is attached to and between proximal landing zone 102 and distal landing zone 106. Landing zones 102, 106 are sometimes called seal fixation zones.

As used herein, the proximal end of a prosthesis such as high metal to vessel ratio landing zone stent-graft 100 is the end closest to the heart via the path of blood flow whereas the distal end is the end furthest away from the heart during deployment. In contrast and of note, the distal end of the delivery system is usually identified to the end that is farthest from the operator (handle) while the proximal end of the delivery system is the end nearest the operator (handle).

For purposes of clarity of discussion, as used herein, the distal end of the delivery system is the end that is farthest from the operator (the end furthest from the handle) while the distal end of the prosthesis is the end nearest the operator (the end nearest the handle), i.e., the distal end of the delivery system and the proximal end of the prosthesis are the ends furthest from the handle while the proximal end of the delivery system and the distal end of the prosthesis are the ends nearest the handle. However, those of skill in the art will understand that depending upon the access location, the prosthesis and delivery system description may be consistent or opposite in actual usage.

Proximal landing zone 102 includes a proximal end 102P and a distal end 102D. Exclusion zone 104 includes a proximal end 104P and a distal end 104D. Distal end 102D of proximal landing zone 102 is attached to proximal end 104P of exclusion zone 104 by an attachment structure 103. Illustratively, attachment structure 103 is stitching, adhesive, thermal bonding, or other attachment between proximal landing zone 102 and exclusion zone 104.

Distal landing zone 106 includes a proximal end 106P and a distal end 106D. Proximal end 106P of distal landing zone 106 is attached to distal end 104D of exclusion zone 104 by an attachment structure 105. Illustratively, attachment structure 105 is stitching, adhesive, thermal bonding, or other attachment between exclusion zone 104 and distal landing zone 106.

High metal to vessel ratio landing zone stent-graft 100 includes a proximal main opening 108 at a proximal end 100P of high metal to vessel ratio landing zone stent-graft 100 and a distal main opening 110 at a distal end 100D of high metal to vessel ratio landing zone stent-graft 100.

Further, high metal to vessel ratio landing zone stent-graft 100 includes a longitudinal axis L. A main lumen 112 is defined by high metal to vessel ratio landing zone stent-graft 100 and extends generally parallel to longitudinal axis L and between proximal main opening 108 and distal main opening 110 of high metal to vessel ratio landing zone stent-graft 100.

In accordance with this embodiment, proximal landing zone 102, exclusion zone 104, and distal landing zone 106 are cylindrical having a substantially uniform diameter D. Stated another way, high metal to vessel ratio landing zone stent-graft 100 has a substantially uniform diameter D. However, in other embodiments, high metal to vessel ratio landing zone stent-graft 100 has a non-uniform diameter.

Proximal landing zone 102 is sometimes called a region, area, or section, that is set off as being distinct from exclusion zone 104. In accordance with this embodiment, proximal landing zone 102 is formed of a high metal to vessel ratio metal mesh.

More particularly, proximal landing zone 102 is a semi-permeable barrier made of connected strands of metal 114, e.g., is a dense cylindrical braided metal mesh. Proximal landing zone 102 includes metal 114 and a plurality of holes 116 through which fluid, e.g., blood, can pass. Generally, proximal landing zone 102 is permeable, sometimes called porous, to fluid, i.e., fluid can pass through proximal landing zone 102 and more particularly, through holes 116. This allows fluid, e.g., blood, to pass through proximal landing zone 102 and nourish, e.g., with oxygen and nutrients, the covered vessel wall. In this manner, hypoxia of the covered vessel wall is avoided. Further, proximal landing zone 102 is permeable to tissue ingrowth.

Holes 116 are generally arranged as an array 118, e.g., a pattern of regularly spaced holes 116 within metal 114. Metal 114 is cylindrical in shape. In one embodiment, array 118 includes holes 116 arranged in both the longitudinal direction 120 and the circumferential direction 122 along proximal landing zone 102.

Longitudinal direction 120 is the direction along proximal landing zone 102 parallel to longitudinal axis L of high metal to vessel ratio landing zone stent-graft 100. Circumferential direction 122 is the direction along the circumference of proximal landing zone 102 in plane perpendicular to longitudinal axis L of high metal to vessel ratio landing zone stent-graft 100. Generally, there are a plurality, e.g., three or more, of holes 116 arranged in both longitudinal direction 120 as well as circumferential direction 122.

The ratio of metal 114 per unit area of proximal landing zone 102 is high, e.g., greater than or equal 30%. This ratio is sometimes called the metal to vessel ratio (or metal to artery ratio) as it defines the percent of the vessel covered with metal 114 per unit area of the vessel. Stated another way, the percentage of proximal landing zone 102 formed by metal 114 is high, e.g., greater than or equal to 30%, and the percentage of proximal landing zone 102 formed of holes 116 is low, e.g., less than or equal to 70%.

Generally, the metal to vessel ratio is defined as the area occupied by metal 114 of proximal landing zone 102 for a unit area of proximal landing zone 102 when in the final configuration. To illustrate, for a X square centimeter (cm²) area of proximal landing zone 102, Y percent is formed of metal 114 whereas Z percent is formed of holes 116, where Y+Z=100. Continuing with this example, Y is the metal to vessel ratio expressed as percent.

To give a specific example for a 40% metal to vessel ratio proximal landing zone 102, for a 1.0 square centimeter area of proximal landing zone 102, 0.4 square centimeters would be covered by metal 114 whereas 0.6 square centimeters would be covered by holes 116. The metal to vessel ratio can be expressed as a fraction, e.g., 0.4 for this example, or as a percentage, e.g., 40% for this example. To convert, the fraction is multiplied by 100 to obtain the percentage.

Although a fixed metal to vessel ratio is set forth, in other embodiments, the metal to vessel ratio of proximal landing zone 120 varies in the longitudinal direction 120 and/or in the circumferential direction 122 along proximal landing zone 102.

As set forth above, the metal to vessel ratio is defined when proximal landing zone 102 is in the final configuration. Proximal landing zone 102 is in the final configuration when in its final unconstrained expanded state, sometimes called at nominal deployment. More particularly, when the diameter of proximal landing zone 102 is approximately equal to the diameter of the vessel in which proximal landing zone 102 is being deployed and proximal landing zone 102 is at its natural unconstrained length at this diameter, proximal landing zone 102 is in its final state. Generally, once deployed within the vessel at its natural unconstrained length as discussed below, proximal landing zone 102 is in the final configuration.

The final configuration should be contrasted to the constrained configuration of proximal landing zone 102. Proximal landing zone 102 is in a constrained configuration when proximal landing zone 102 is constrained to a reduced diameter, e.g., within a delivery sheath. Further, proximal landing zone 102 is in a constrained configuration when proximal landing zone 102 is constrained to a reduced or expanded length, e.g., by longitudinally compressing or expanding proximal landing zone 102. When in the constrained configuration, either in length, diameter, or both, holes 116 are collapsed resulting in a much higher metal to vessel ratio for proximal landing zone 102 than when proximal landing zone 102 is in its final configuration.

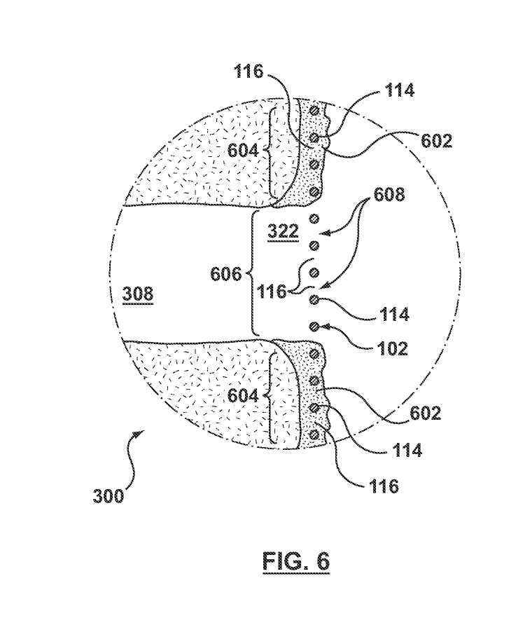

As discussed further below, e.g., in reference to FIGS. 5-6, the metal to vessel ratio of proximal landing zone 102 is sufficiently high to encourage tissue ingrowth around proximal landing zone 102. However, the metal to vessel ratio of proximal landing zone 102 is sufficiently low to ensure adequate perfusion of branch vessel(s) through proximal landing zone 102.

Generally, the metal to vessel ratio of proximal landing zone 102 is within the range of 30 percent to 80 percent (30-80%), more suitably within the range of 35 percent to 60 percent (35-60%). In one particular embodiment, the metal to vessel ratio is 40 percent (40%).

In one embodiment, proximal landing zone 102 is formed of balloon expandable and/or self-expanding metal, e.g., e.g., formed of Nitinol or stainless steel. As set forth above, in one particular embodiment, proximal landing zone 102 is formed of interwoven metal strands forming a metal mesh.

However, in other embodiments, proximal landing zone 102 is a laser cut or etched stent. For example, a cylindrical tube of metal, e.g., Nitinol, is cut with a laser and/or by etching to form holes 116 therein thus forming proximal landing zone 102. The cylindrical tube of metal can be formed from a metal sheet that is bent and welded in one embodiment.

Exclusion zone 104 is sometimes called a region, area, or section, that is set off as being distinct from proximal landing zone 102 and distal landing zone 106. In accordance with this embodiment, exclusion zone 104 includes a cylindrical piece of graft material 124, e.g., graft cloth formed of polyester, Dacron, ePTFE (expanded Polytetrafluoroethylene), and/or polyurethane material.

Further, exclusion zone 104 includes one or more support structures 126. Illustratively, support structures 126 are self-expanding stent rings, e.g., formed of Nitinol. Support structures 126 are attached to graft material 124, e.g., with stitching, adhesive, thermal bonding, or other attachment between support structures 126 and graft material 124. In another embodiment, exclusion zone 104 is formed without support structures 126, which are thus optional. In FIG. 2 and the following figures, support structures 126 are not illustrated for simplicity although it is to be understood that support structures 126 would be present depending upon the embodiment.

Generally, exclusion zone 104 is a barrier to fluid, e.g., blood. More particularly, exclusion zone 104 is impermeable to fluid, i.e., fluid cannot pass through exclusion zone 104 to any significant degree.

In one embodiment, distal landing zone 106 is substantially identical in structure to proximal landing zone 102. Distal landing zone 106 includes metal 114A, holes 116A arranged in an array 118A in a manner similar or identical to metal 114, holes 116, and array 118 of proximal landing zone 102, respectively. Distal landing zone 106 has the same metal to vessel ratio as proximal landing zone 102 in this embodiment.

In another embodiment, distal landing zone 106 has a metal to vessel ratio different than proximal landing zone 102. For example, branch vessel do not need to be perfused through distal landing zone 106 and so distal landing zone 106 is formed with a metal to vessel ratio higher than proximal landing zone 102 to promote tissue ingrowth without risk of occluding branch vessels.

In yet another embodiment, high metal to vessel ratio landing zone stent-graft 100 is formed without distal landing zone 106, which is thus an optional structure.

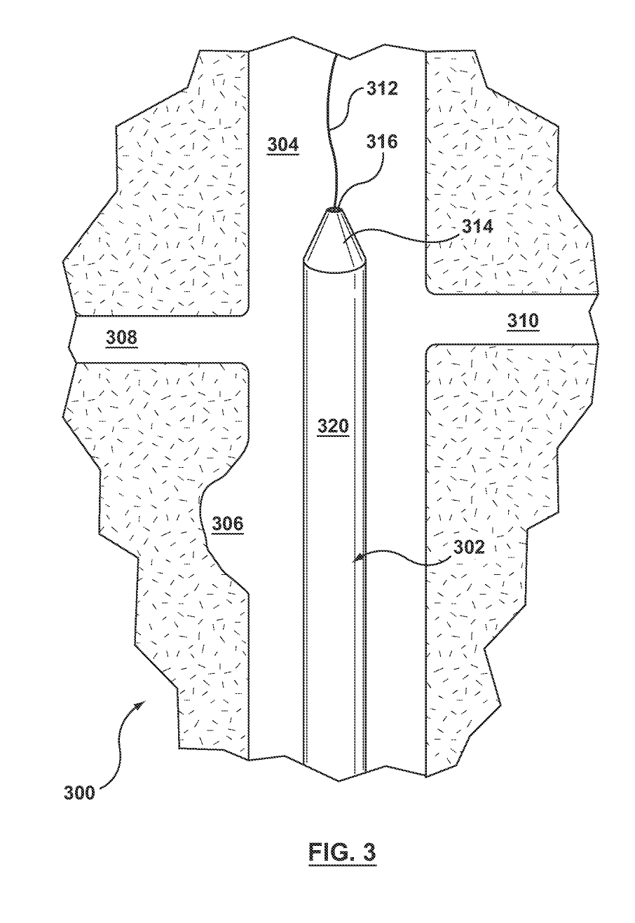

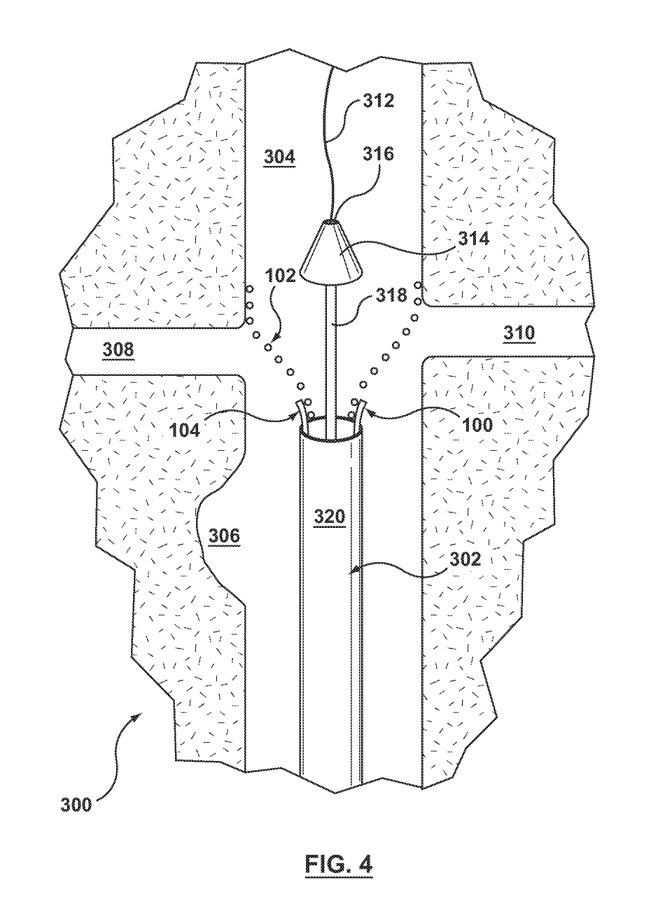

FIG. 3 is a cross-sectional view of a vessel assembly 300 including a delivery system 302 including high metal to vessel ratio landing zone stent-graft 100 of FIGS. 1 and 2 in accordance with one embodiment. FIG. 4 is a cross-sectional view of vessel assembly 300 including delivery system 302 at a later stage of deploying high metal to vessel ratio landing zone stent-graft 100 of FIGS. 1 and 2 in accordance with one embodiment.

Referring now to FIGS. 3 and 4 together, a main vessel 304, e.g., the aorta, includes an aneurysm 306. High metal to vessel ratio landing zone stent-graft 100, sometimes called a prosthesis, is deployed into main vessel 304 to exclude aneurysm 306 using delivery system 302.

Emanating from main vessel 304 is a first branch vessel 308 and a second branch vessel 310, sometimes called visceral branches of the abdominal aorta. The location of branch vessels 308, 310 vary from patient to patient. Examples of branch vessels include the renal arteries (RA), the superior mesenteric artery (SMA), the brachiocephalic artery, the left subclavian artery, the left common carotid, the celiac trunk, and the hypogastric artery.

Delivery system 302 is advanced to the location of aneurysm 306, e.g., over a guidewire 312, for example as illustrated in FIG. 3. Delivery system 302 includes a tapered tip 314 that is flexible and able to provide trackability in tight and tortuous vessels. Tapered tip 314 includes a lumen 316 allowing for passage of guidewire 312 in accordance with this embodiment. In one embodiment, delivery system 302 includes radiopaque marker(s) that allow visualization of delivery system 302.

To deploy high metal to vessel ratio landing zone stent-graft 100, an inner member 318 of delivery system 302 including tapered tip 314 mounted thereon is held stationary while an outer sheath 320 of delivery system 302 is withdrawn, for example, as illustrated in FIG. 4. High metal to vessel ratio landing zone stent-graft 100 is radially constrained by outer sheath 320 around inner member 318. Inner member 318 includes a stent stop or other features to prevent high metal to vessel ratio landing zone stent-graft 100 from moving back as outer sheath 320 is withdrawn.

As outer sheath 320 is withdrawn, high metal to vessel ratio landing zone stent-graft 100 is gradually exposed from proximal end 100P to distal end 100D of high metal to vessel ratio landing zone stent-graft 100. The exposed portion of high metal to vessel ratio landing zone stent-graft 100 radially expands to be in conforming surface contact with main vessel 304. More particularly, high metal to vessel ratio landing zone stent-graft 100 opposes the walls of main vessel 304 thus securing high metal to vessel ratio landing zone stent-graft 100 in place.

In one embodiment, high metal to vessel ratio landing zone stent-graft 100 is self-expanding and thus self expands upon being released from outer sheath 320. However, in other embodiments, high metal to vessel ratio landing zone stent-graft 100 is expanded with a balloon or other expansion device.

Although a particular delivery system 302 is illustrated in FIGS. 3, 4 and discussed above, in light of this disclosure, those of skill in the art will understand that any one of a number of delivery systems can be used to deploy high metal to vessel ratio landing zone stent-graft 100 and the particular delivery system used is not essential to this embodiment.

FIG. 5 is a cross-sectional view of vessel assembly 300 of FIGS. 3-4 after deployment of high metal to vessel ratio landing zone stent-graft 100 of FIGS. 1 and 2 in accordance with one embodiment. Referring now to FIG. 5, high metal to vessel ratio landing zone stent-graft 100 is in conforming surface contact with main vessel 304. High metal to vessel ratio landing zone stent-graft 100 is deployed such that proximal landing zone 102 covers, sometimes called jails, ostai (plural of ostium) 322, 324 of branch vessels 308, 310, respectively.

However, as proximal landing zone 102 is permeable, blood flows from main vessel 304 through proximal landing zone 102 and into branch vessels 308, 310 thus perfusing branch vessels 308, 310. In one embodiment, branch vessels 308, 310 are continuously perfused during the entire procedure of deploying high metal to vessel ratio landing zone stent-graft 100.

Further, deployment of high metal to vessel ratio landing zone stent-graft 100 is relatively simple thus minimizing the complexity and thus risk of deploying high metal to vessel ratio landing zone stent-graft 100. More particularly, as the entire proximal landing zone 102 is permeably, high metal to vessel ratio landing zone stent-graft 100 is deployed without having to rotationally position high metal to vessel ratio landing zone stent-graft 100 to be aligned with branch vessels 308, 310.

Further, proximal landing zone 102 is deployed with fixation and sealing to main vessel 304 superior to aneurysm 306, e.g., to healthy tissue of main vessel 304 adjacent branch vessels 308, 310. This minimizes the risk of migration of high metal to vessel ratio landing zone stent-graft 100. Further, this allows fixation and sealing of proximal landing zone 102 to healthy tissue even when aneurysm 306 has a short neck, i.e., when the distance between aneurysm 306 and branch vessels 308, 310 is relatively small, as well as when aneurysm 306 has a highly angulated neck.

Further, exclusion zone 104 covers and excludes aneurysm 306. More particularly, once high metal to vessel ratio landing zone stent-graft 100 is anchored within main vessel 304, blood flows through main lumen 112 and more generally through exclusion zone 104 thus excluding aneurysm 306. By forming exclusion zone 104 of graft material which is impermeable to blood, excellent exclusion of aneurysm 306 is achieved.

Further, distal landing zone 106 is deployed with fixation and sealing to main vessel 304 inferior to aneurysm 306, e.g., to healthy tissue of main vessel 304. This further facilitates exclusion of aneurysm 306 while at the same time minimizes the risk of migration of high metal to vessel ratio landing zone stent-graft 100.

As set forth, in one embodiment, high metal to vessel ratio landing zone stent-graft 100 is formed without distal landing zone 106. In other examples, high metal to vessel ratio landing zone stent-graft 100 is a bifurcated stent-graft, e.g., exclusion zone 104 is bifurcated to extend into the iliac arteries.

As discussed above, by forming proximal landing zone 102 to have a high metal to vessel ratio, branch vessels 308, 310 are adequately perfused through proximal landing zone 102 while at the same time tissue ingrowth of main vessel 304 into proximal landing zone 102 is encouraged. Further, by forming exclusion zone 104 of graft material which is impermeable to blood, excellent exclusion of aneurysm 306 is achieved.

FIG. 6 is an enlarged cross-sectional view of the region VI of FIG. 5 illustrating tissue 602 ingrowth into proximal landing zone 102 of high metal to vessel ratio landing zone stent-graft 100. For example, FIG. 6 illustrates ingrowth of tissue 602 after a period of time, e.g., weeks or months, after the deployment of high metal to vessel ratio landing zone stent-graft 100 into main vessel 304. Ingrowth of tissue 602 is sometimes called endovascular repaving of the seal zone of main vessel 304 or neointimal overgrowth.

Referring now to FIGS. 5 and 6 together, once deployed, proximal landing zone 102 includes a fixation region 604 and a perfusion region 606. Fixation region 604 is the region of proximal landing zone 102 in direct contact with main vessel 304. Perfusion region 606 is the region of proximal landing zone 102 covering ostium 322 of branch vessel 308. Perfusion region 606 also includes the region of proximal landing zone 102 covering ostium 324 of branch vessel 310, which is not illustrated in FIG. 6, but see FIG. 5.

After deployment of high metal to vessel ratio landing zone stent-graft 100, tissue 602 of main vessel 304 grows through holes 116 of fixation region 604 of proximal landing zone 102. Tissue 602 encases, sometimes called encloses or encapsulates, metal 114 of fixation region 604 of proximal landing zone 102.

This ingrowth of tissue 602 provides secure fixation and sealing of proximal landing zone 102 to main vessel 304 and generally of high metal to vessel ratio landing zone stent-graft 100 to main vessel 304. By providing secure fixation and sealing of proximal landing zone 102 to main vessel 304, the risk of endoleaks into aneurysm 306 and migration of high metal to vessel ratio landing zone stent-graft 100 is minimized. Further, the ingrowth of tissue 602 restricts expansion of aneurysm 306 into this region of main vessel 304.

Further, as illustrated in FIG. 6, tissue 602 does not grow over perfusion region 606 of proximal landing zone 102. More particularly, blood flows as indicated by the arrows 608 through holes 116 of perfusion region 606 to perfuse branch vessel 308. Further, this blood flow prevents tissue overgrowth on perfusion region 606 thus avoiding occlusion of branch vessel 308 and the associated complications.

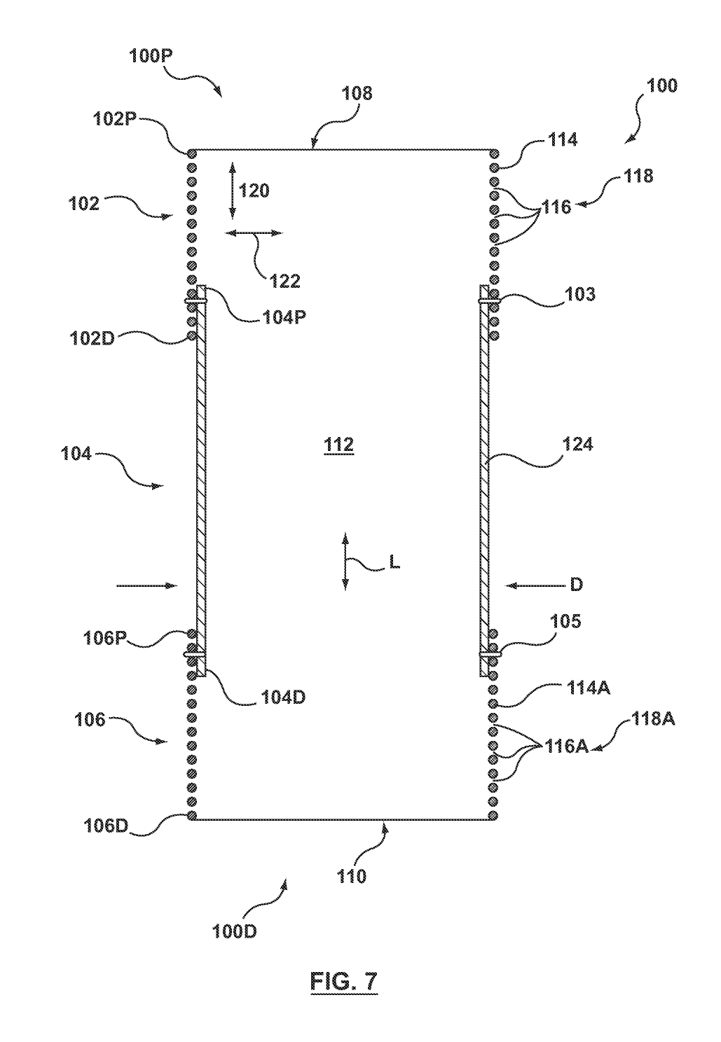

FIG. 7 is a cross-sectional view of high metal to vessel ratio landing zone stent-graft 100 of FIG. 1 in accordance with another embodiment. High metal to vessel ratio landing zone stent-graft 100 of FIG. 7 is similar to high metal to vessel ratio landing zone stent-graft 100 of FIG. 2 except that landing zones 102, 106 overlap the outside of exclusion zone 104 in FIG. 7 yet overlap the inside of exclusion zone 104 in FIG. 2.

More particularly, in FIG. 7, exclusion zone 104 is inside of proximal landing zone 102 at the overlap of proximal landing zone 102 and exclusion zone 104. In this manner, the entire proximal landing zone 102 is exposed to the outside thus allowing tissue overgrowth of the entire proximal landing zone 102.

Similarly, exclusion zone 104 is inside of distal landing zone 106 at the overlap of distal landing zone 106 and exclusion zone 104. In this manner, the entire distal landing zone 106 is exposed to the outside thus allowing tissue overgrowth of the entire distal landing zone 106.

In another embodiment, exclusion zone 104 includes two or more layers, e.g., of graft material. In accordance with this embodiment, proximal landing zone 102 and/or distal landing zone 106 are sandwiched between layers of exclusion zone 104. Generally, proximal landing zone 102 and/or distal landing zone 106 are located outside of exclusion zone 104, inside of exclusion zone 104, or sandwiched between layers of exclusion zone 104 at the overlap with exclusion zone 104.

This disclosure provides exemplary embodiments. The scope is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A prosthesis comprising:
a proximal landing zone comprising metal comprising an array of holes therein, wherein a metal to vessel ratio of the proximal landing zone when in the final configuration is sufficiently high to encourage tissue ingrowth around the proximal landing zone yet is sufficiently low to ensure perfusion of a branch vessel through the proximal landing zone; and
an exclusion zone attached to the proximal landing zone, the exclusion zone comprising graft material.

2. The prosthesis of claim 1 wherein the metal to vessel ratio is within the range of 30 percent to 80 percent.

3. The prosthesis of claim 1 wherein the metal to vessel ratio is within the range of 35 percent to 60 percent.

4. The prosthesis of claim 1 wherein the array comprises the holes regularly spaced as a pattern within the metal.

5. The prosthesis of claim 1 wherein the array comprises three or more of the holes arranged in a longitudinal direction of the proximal landing zone.

6. The prosthesis of claim 1 wherein the array comprises three or more holes arranged in a circumferential direction of the proximal landing zone.

7. The prosthesis of claim 1 wherein the metal to vessel ratio is the percentage of the proximal landing zone covered by the metal when the proximal landing zone is in the final configuration.

8. The prosthesis of claim 1 wherein a percentage of the proximal landing zone covered by the metal plus a percentage of the proximal landing zone covered by the holes equals 100 percent.

9. The prosthesis of claim 1 further comprising a distal landing zone attached to the exclusion zone.

10. The prosthesis of claim 9 wherein the distal landing zone comprises a metal comprising an array of holes therein, wherein the metal to vessel ratio of the distal landing zone is sufficiently high to encourage tissue ingrowth around the distal landing zone.

11. The prosthesis of claim 9 further comprising an attachment structure for attaching the distal landing zone to the exclusion zone.

12. The prosthesis of claim 1 further comprising an attachment structure for attaching the exclusion zone to the proximal landing zone.

13. The prosthesis of claim 1 wherein the exclusion zone further comprises one or more support structures attached to the graft material.

14. The prosthesis of claim 1 wherein the metal comprises a cylindrical braided metal mesh.

15. The prosthesis of claim 1 wherein the metal comprises a cut cylindrical tube.

16. A delivery system comprising:
a prosthesis comprising:
a proximal landing zone comprising metal comprising an array of holes therein, wherein the metal to vessel ratio of the proximal landing zone when in the final configuration is sufficiently high to encourage tissue ingrowth around the proximal landing zone yet is sufficiently low to ensure perfusion of a branch vessel through the proximal landing zone; and
an exclusion zone attached to the proximal landing zone, the exclusion zone comprising graft material; and
an outer sheath radially constraining the prosthesis.

17. The delivery system of claim 16 further comprising an inner member, the prosthesis being radially constrained around the inner member by the outer sheath.

18. A method comprising:
deploying a prosthesis into a main vessel comprising:
covering an ostium of a branch vessel emanating from the main vessel with a proximal landing zone of the prosthesis, wherein a metal to vessel ratio of the proximal landing zone when deployed is sufficiently high to encourage tissue ingrowth around the proximal landing zone yet is sufficiently low to ensure perfusion of the branch vessel through the proximal landing zone; and
covering an aneurysm of the main vessel with an exclusion zone of the prosthesis, the exclusion zone comprising graft material.

19. The method of claim 18 wherein the main vessel is the abdominal aorta.

20. The method of claim 18 wherein the deploying comprises retracting an outer sheath to expose the prosthesis, wherein the prosthesis self-expands upon being exposed.

21. The method of claim 18 wherein fluid flows from the main vessel through the proximal landing zone and into the branch vessel.

22. The method of claim 18 wherein the branch vessel is continuously perfused during the deploying a prosthesis.

23. The method of claim 18 wherein the proximal landing zone is deployed superior to the aneurysm.

24. The method of claim 18 wherein the exclusion zone excludes the aneurysm.

25. The method of claim 18 wherein the deploying a prosthesis further comprises deploying a distal landing zone of the prosthesis inferior to the aneurysm.

26. The method of claim 25 wherein the distal landing zone comprises a metal comprising an array of holes therein, wherein the metal to vessel ratio of the distal landing zone is sufficiently high to encourage tissue ingrowth around the distal landing zone.

27. The method of claim 18 wherein the proximal landing zone comprises:
a fixation region in direct contact with the main vessel; and
a perfusion region covering the ostium of the branch vessel.

28. The method of claim 27 wherein tissue of the main vessel encases the fixation region.

29. The method of claim 27 wherein fluid flow through the perfusion region prevents tissue overgrowth on the perfusion region.
